(19) United States
(12) Patent Application Publication
(10) Pub. No.: US 2025/0084127 A1
(43) Pub. Date: Mar. 13, 2025

(54) EFFICIENT CHEMO-ENZYMATIC SYNTHESIS METHOD FOR CYCLIC PEPTIDE

(71) Applicant: NATIONAL UNIVERSITY CORPORATION HOKKAIDO UNIVERSITY, Hokkaido (JP)

(72) Inventors: Toshiyuki WAKIMOTO, Sapporo-shi, Hokkaido (JP); Kenichi MATSUDA, Sapporo-shi, Hokkaido (JP); Masakazu KOBAYASHI, Sapporo-shi, Hokkaido (JP)

(73) Assignee: NATIONAL UNIVERSITY CORPORATION HOKKAIDO UNIVERSITY, Hokkaido (JP)

(21) Appl. No.: 18/701,365

(22) PCT Filed: Oct. 17, 2022
(86) PCT No.: PCT/JP2022/038513

§ 371 Date: Nov. 21, 2024

(30) Foreign Application Priority Data
Oct. 18, 2021 (JP) 2021-170218
Feb. 21, 2022 (JP) 2022-024966

(51) Int. Cl. C07K 7/50; C12N 9/16

(52) U.S. Cl. CPC C07K 7/50; C12N 9/16; C12Y 301/03005

(57) ABSTRACT

A method for producing a cyclic peptide, comprising using a penicillin-binding protein-type thioesterase (PBP-type TE) or a tyrocidine synthase TycC thioesterase domain (TycC-TE) as a catalyst, wherein a diol as a leaving group is attached to a carboxyl group of a C-terminal residue of a substrate; and a method for producing a peptide serving as the substrate using a solid phase carrying a diol.

### TECHNICAL FIELD

The present invention relates to a method for producing a cyclic peptide.

### RELATED APPLICATIONS

This application claims the benefits of the priorities of Japanese Patent Application No. 2021-170218 filed Oct. 18, 2021, and Japanese Patent Application No. 2022-024966 filed Feb. 21, 2022, to the Japanese Patent Office. The entire contents of these applications are incorporated herein by reference.

### BACKGROUND ART

Peptides, which are members of a major compound class of middle-molecule pharmaceutical seeds, are improved in metabolic stability, membrane permeability and target specificity by cyclization. However, a peptide cyclization reaction by an organic synthetic approach is difficult to control. In addition, the peptide cyclization reaction has the following problems: by-products difficult to separate are produced and a large amount of an organic solvent is consumed (Non Patent Literature 1). Conversely, an enzyme catalyzes a peptide cyclization reaction efficiently under mild conditions. Therefore, the enzyme will bring a novel synthetic method having high friendliness to the environment.

Up to the present, the inventors have found a novel peptide cyclase penicillin-binding protein-type thioesterase (PBP-type TE) in soil bacteria (actinomycetes) (Non Patent Literature 2, Patent Literature 1). This enzyme can catalyze synthesis of a short-chain cyclic peptide having about 10 residues or less, where chemical cyclization tends to be difficult. In addition, since this enzyme has broad substrate selectivity, it has high potential as a biocatalyst.

### CITATION LIST

### Patent Literature

Patent Literature 1: WO2019/216248

### Non Patent Literatures

Non Patent Literature 1: White, C., Yudin, A. Nat. Chem. 3, 509-524 (2011).
Non Patent Literature 2: Matsuda, K. et al., Nat. Catal. 3, 507-515 (2020).

### SUMMARY OF INVENTION

### Technical Problem to be Solved by the Invention

There is a need for the development of an efficient and simple method for enzymatic synthesis of a cyclic peptide, and a novel cyclic peptide. The PBP-type TE has a high potential as a biocatalyst, however a leaving group is required for the C terminal of a substrate for catalysis by this enzyme. For this purpose, it is necessary to find a material for a leaving group that can enhance cyclization efficiency at a low cost. Furthermore, it is necessary to find an enzyme capable of cyclizing a substrate having such a leaving group attached thereto. Moreover, it is desired to develop a method for efficiently and simply synthesizing a substrate having such a leaving group attached thereto.

### Means to Solve the Problem

The present inventors have conducted intensive studies to solve the above problems. As a result, they have found that a PBP-type TE and a thioesterase domain of a tyrocidine synthase TycC (TycC-TE) can adopt an inexpensive ethylene glycol (hereinafter sometimes referred to as “EG”) as a leaving group and catalyzes a peptide cyclization reaction. The present inventors also developed a new synthetic route of a substrate peptide, where a peptide is elongated from EG previously carried on a solid phase. Based on these findings and developments, the present invention was accomplished.

That is, the present invention provides the followings.

(1) A method for producing a cyclic peptide, comprising using a PBP-type TE or a TycC-TE as a catalyst, wherein a diol as a leaving group is attached to a carboxyl group of a C terminal residue of a substrate.

(2) The method according to (1), wherein the leaving group is EG or an analogue thereof.

(3) The method according to (2), wherein the leaving group is EG.

(4) The method according to any one of (1) to (3), wherein the PBP-type TE is used as the catalyst.

(5) The method according to (4), wherein the PBP-type TE is an enzyme having an amino acid sequence represented by SEQ ID NO: 2 or a mutant enzyme thereof, and wherein the mutant enzyme has any one of the following amino acid sequences:
(a) an amino acid sequence having an identity of 38% or more to the amino acid sequence represented by SEQ ID NO: 2;
(b) an amino acid sequence having a substitution, deletion, insertion or addition of one, several or several tens of amino acids in the amino acid sequence represented by SEQ ID NO: 2; or
(c) an amino acid sequence encoded by a base sequence that hybridizes with a base sequence complementary to a base sequence represented by SEQ ID NO: 1 under a stringent condition, and has a peptide cyclization activity equivalent to or higher than that of the enzyme having the amino acid sequence represented by SEQ ID NO: 2.

(6) The method according to (4), wherein the PBP-type TE is an enzyme having an amino acid sequence represented by SEQ ID NO: 14 or a mutant enzyme thereof, and wherein the mutant enzyme has any one of the following amino acid sequences:
(a) an amino acid sequence having an identity of 35% or more to the amino acid sequence represented by SEQ ID NO: 14,
(b) an amino acid sequence having a substitution, deletion, insertion or addition of one, several or several tens of amino acids in the amino acid sequence represented by SEQ ID NO: 14, or
(c) an amino acid sequence encoded by a base sequence that hybridizes with a base sequence complementary to a base sequence represented by SEQ ID NO: 13 under a stringent condition, and has a peptide cyclization activity equivalent to or higher than that of the enzyme having the amino acid sequence represented by SEQ ID NO: 14.

(7) The method according to any one of (1) to (3), wherein the TycC-TE is used as the catalyst.

(8) The method according to (7), wherein the TycC-TE is an enzyme having an amino acid sequence represented by SEQ ID NO: 7 or a mutant enzyme thereof; and wherein the mutant enzyme has any one of the following amino acid sequences:
(a) an amino acid sequence having an identity of 35% or more to the amino acid sequence represented by SEQ ID NO: 7;
(b) an amino acid sequence having a substitution, deletion, insertion or addition of one, several or several tens of amino acids in the amino acid sequence represented by SEQ ID NO: 7; or
(c) an amino acid sequence encoded by a base sequence that hybridizes with a base sequence complementary to a base sequence represented by SEQ ID NO: 6 under a stringent condition, and has a peptide cyclization activity equivalent to or higher than that of the enzyme having the amino acid sequence represented by SEQ ID NO: 7.

(9) The method according to any one of (1) to (8), wherein the substrate is obtained by a synthesis method comprising the following steps:
(i) elongating a peptide from a diol carried on a solid phase; and
(ii) cleaving the peptide to which the diol is bound from the solid phase.

(10) The method according to (9), wherein the diol is EG or an analogue thereof.

(11) The method according to (10), wherein the diol is EG.

(12) A kit for producing a cyclic peptide, comprising a PBP-type TE or a TycC-TE.

(13) The kit according to (12), further comprising a substrate having a diol as a leaving group attached to a carboxyl group of a C-terminal residue thereof, or a means for producing the substrate.

### Effects of Invention

According to the present invention, there is provided a method enabling cyclization of a peptide by enzymatic synthesis using a PBP-type TE or a TycC-TE and a peptide having a diol as a leaving group as a substrate, in a head-to-tail style. According to the present invention, there is further provided a method for efficiently and simply producing a substrate peptide having a diol as a leaving group. Of the diols, particularly EG is an extremely inexpensive substance. Thus, it is possible to produce a substrate at an extremely low cost. As a result, the production cost for a cyclic peptide can be reduced. In the present invention, since two types of enzymes, a PBP-type TE and a TycC-TE, can be used as a cyclase, it is possible to produce various types of relatively short-chain cyclic peptides chemical synthesis of which has been difficult. Use of the present invention makes it possible to develop and produce novel medicines, physiologically active substances, biomaterials and the like.

### BRIEF DESCRIPTION OF DRAWINGS

FIG. 1 shows a synthesis scheme of a surugamide B precursor (substrate) having EG as a leaving group and an LC-MS chart of the substrate obtained.

FIG. 2 shows a cyclization reaction scheme of a surugamide B precursor by an enzyme SurE; cyclization reaction conditions; an LC-MS chart (upper) of a cyclization product using SurE; and an LC-MS chart (lower) of a cyclization product using SurE boiled.

FIG. 3 shows a cyclization reaction scheme of a seco-desprenylagaramide C (provided with EG as a leaving group) by a SurE mutant; cyclization reaction conditions; LC-MS analysis conditions; an LC-MS chart (upper) of a cyclization product using the SurE mutant; and an LC-MS chart (lower) of a reaction product in a SurE mutant-free system.

FIG. 4 shows a cyclization reaction scheme of a seco-wollamide B1 (provided with EG as a leaving group) by an enzyme, WolJ; cyclization reaction conditions; LC-MS analysis conditions, an LC-MS chart (upper) of a cyclization product using the WolJ; and an LC-MS chart (lower) of a reaction product in a WolJ-free system.

FIG. 5 shows reverse-phase HPLC charts of cyclization products of a substrate having PEG within the sequence when enzyme SurE was used; reverse-phase HPLC analysis conditions; the structure of the substrate; and the structure of a product. In the charts, +SurE shows the results of an enzyme-containing system and −SurE shows the results of an enzyme-free system.

FIG. 6 shows the outline of the synthesis of a tyrocidine A precursor; a cyclization reaction scheme of the tyrocidine A precursor by an enzyme TycC-TE; cyclization reaction conditions; LC-MS analysis conditions; an LC-MS chart (upper) of a cyclization product using a TycC-TE; and an LC-MS chart (lower) of a reaction product in a TycC-TE-free system.

FIG. 7 shows the sequences of SEQ ID NOS: 1 and 2.

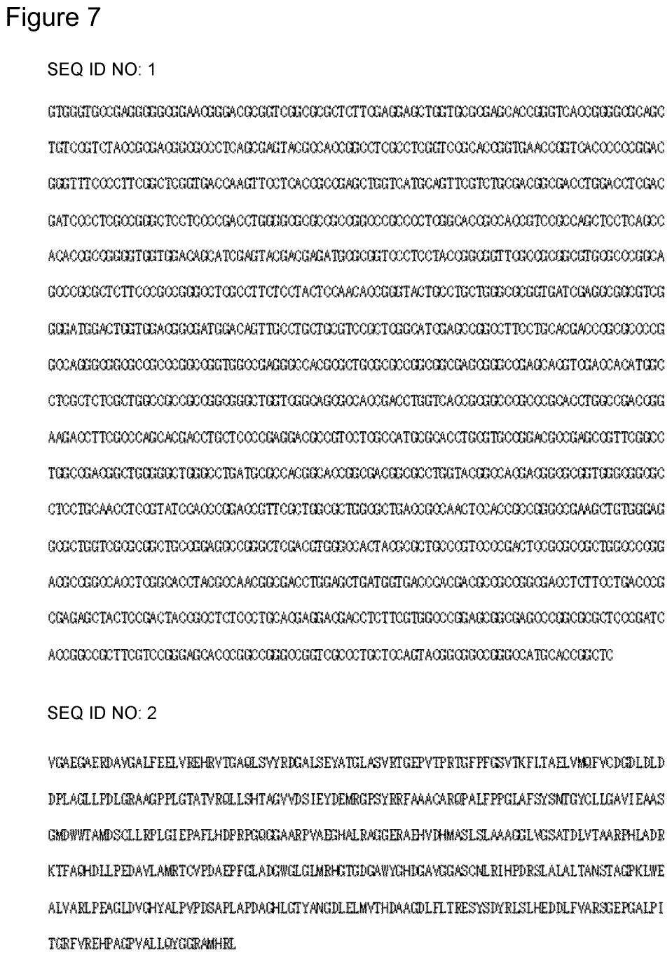

FIG. 8 shows the sequences of SEQ ID NOs: 3 to 6.

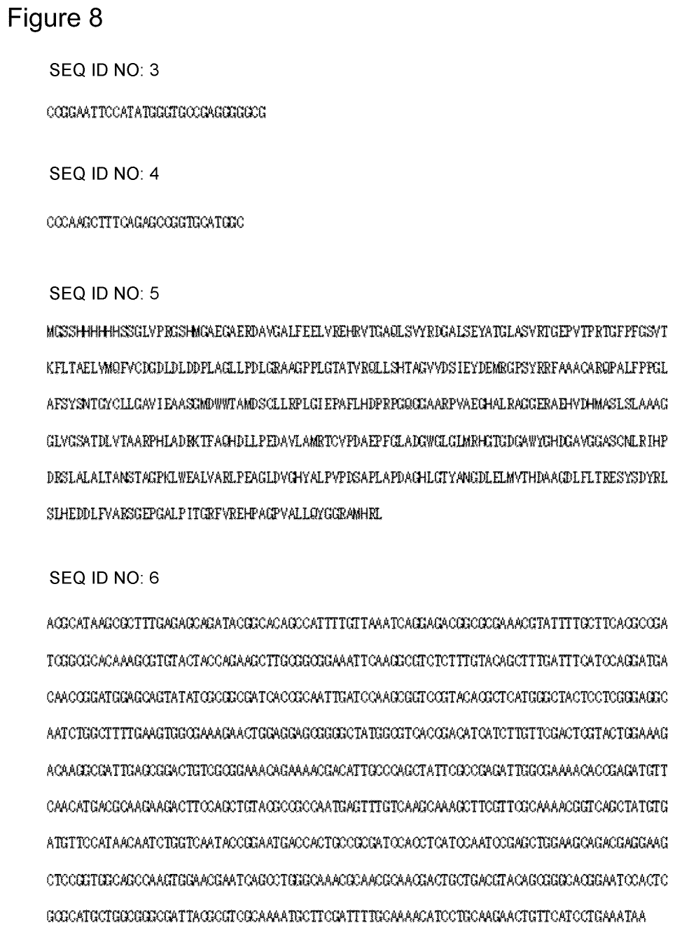

FIG. 9 shows the sequences of SEQ ID NOS: 7 to 9.

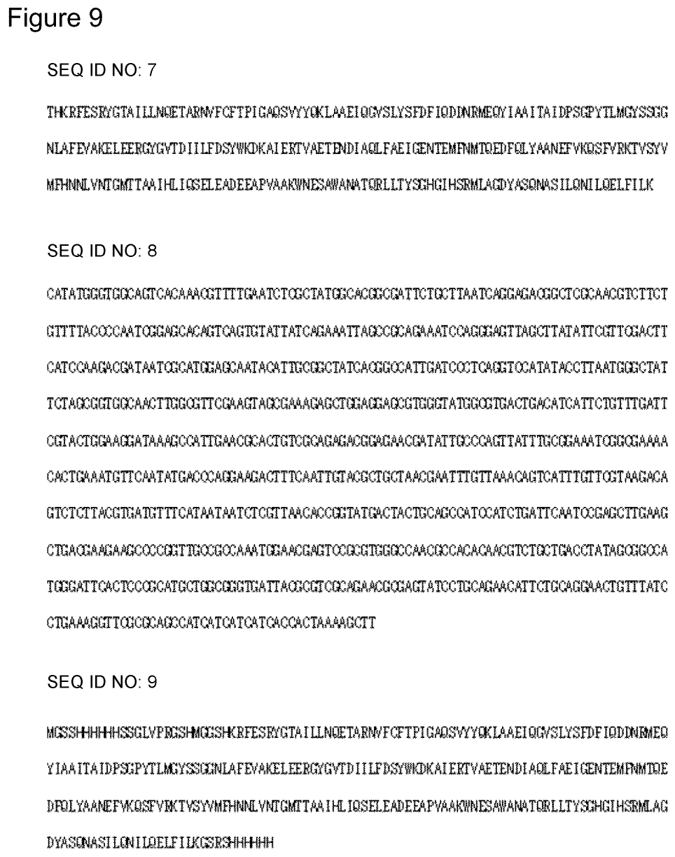

FIG. 10 shows the sequences of SEQ ID NOS: 10 to 12.

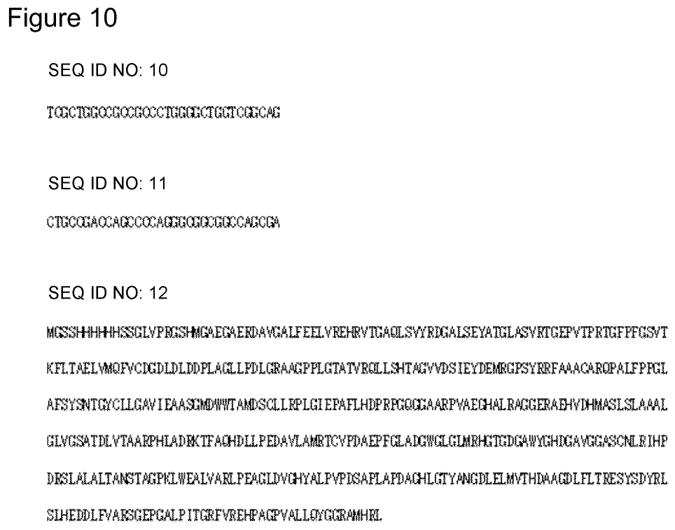

FIG. 11 shows the sequence of SEQ ID NO: 13.

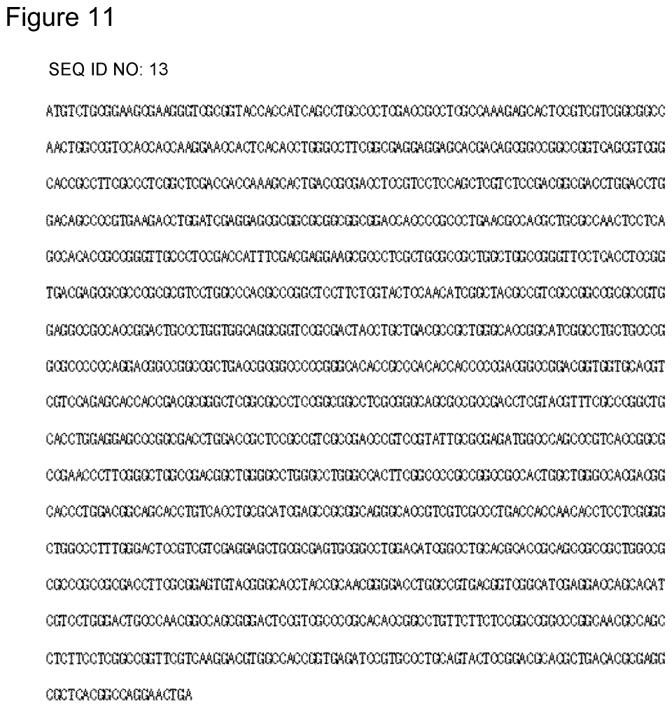

FIG. 12 shows the sequences of SEQ ID NOS: 14 and 15.

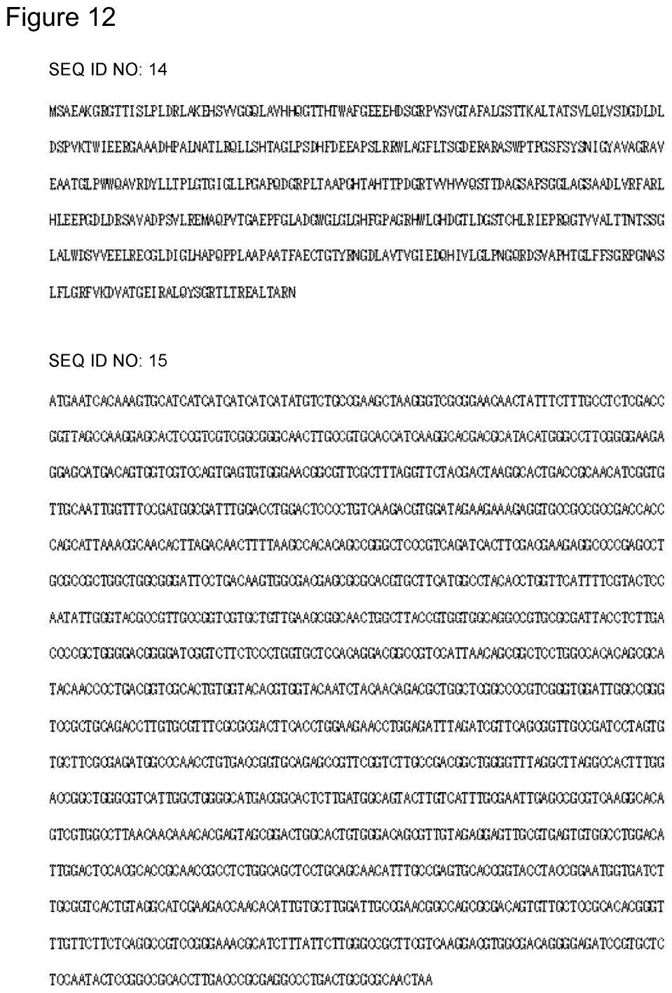

FIG. 13 shows the sequence of SEQ ID NO: 16.

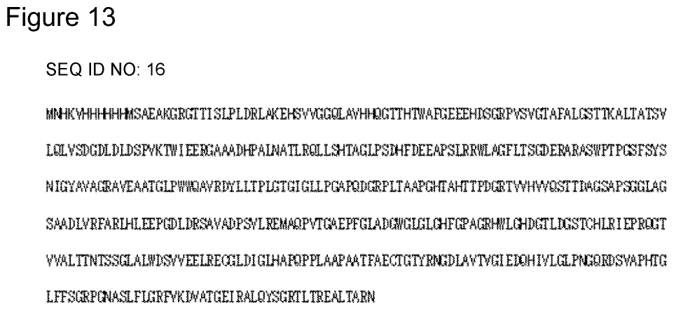

### DESCRIPTION OF EMBODIMENTS

In one aspect, the present invention provides a method for producing a cyclic peptide, comprising using a PBP-type TE or a TycC-TE as a catalyst, wherein a diol as a leaving group is attached to a carboxyl group of a C terminal residue of a substrate.

In one embodiment of this aspect, the catalyst is a PBP-type TE. The PBP-type TE belongs to a group of enzymes found in soil bacteria (actinomycetes) and is characterized by mainly catalyzing a cyclization reaction of a short linear peptide having about 15 residues or less, for example, about 10 residues, and exhibiting a broad substrate selectivity (for PBP-type TE, see, for example, WO2019/216248 and Matsuda, K. et al., Nat. Catal. 3, 507-515 (2020)). Examples of the PBP-type TE include, but are not limited to, SurE, WolJ and Nsm16.

In the present invention, the PBP-type TE is an enzyme that can cyclize a peptide substrate having a diol as a leaving group attached to the carboxyl group of the C-terminal residue, in a head-to-tail mode. An organism from which the PBP-type TE to be used in the present invention is derived is not particularly limited, and the organism is preferably a bacterium, more preferably, a soil bacterium, and further preferably, an actinomycete. Examples of the PBP-type TE derived from actinomycetes include, but are not limited to, enzymes derived from actinomycetes of the genus Streptomyces and the genus Goodfellowiella. The PBP-type TE may be derived from bacteria other than actinomycetes. The PBP-type TE including SurE and WolJ (described later) can be obtained by using a known cloning method.

In the present invention, an enzyme SurE among of the PBP-type TEs, is preferably used. SurE is a PBP-type TE that is possessed by Streptomyces albidoflavus NBRC 12854. The base sequence of DNA encoding SurE is represented by SEQ ID NO: 1. The amino acid sequence of SurE is represented by SEQ ID NO: 2. In the present invention, an enzyme having the amino acid sequence represented by SEQ ID NO: 2 or an enzyme having the amino acid sequence encoded by the base sequence represented by SEQ ID NO: 1 is preferably used. Also, in the present invention, as the PBP-type TE, for example, WolJ derived from Streptomyces sp. MST-110588 and Nsm16 derived from Streptomyces noursei NBRC 15452, etc. may be used.

A PBP-type TE mutant, for example, a SurE mutant may be used in the present invention. The SurE mutant will be explained below. In the specification, unless otherwise specified, the term “SurE” includes its mutants.

A SurE mutant having a cyclization activity equivalent to or higher than that of SurE is preferably used in the present invention. The cyclization activity equivalent to or higher than that of SurE refers to a cyclization activity of about 50% or more, preferably about 70% or more, more preferably about 80% or more, and still more preferably about 90% or more of the cyclization activity of SurE. The cyclization activity of an enzyme can be measured by reacting a substrate and the enzyme and analyzing a product thereof. For example, cyclization activity may be measured by reacting a substrate and an enzyme, subjecting the resultant reaction mixture to LC-MS analysis and measuring the amount of a cyclized product, with reference to the procedure described in Examples of the specification.

Specific embodiments of the SurE mutant include, but are not limited to, enzymes having an amino acid sequence having an identity of about 35% or more, for example, about 38% or more, preferably about 50% or more, more preferably about 70% or more (for example, 75% or more, 80% or more, and 85% or more), and still more preferably about 90% or more (for example, 91% or more, 92% or more, 93% or more, 94% or more, 95% or more, 96% or more, 97% or more, 98% or more, 99% or more, and 99.5% or more) to the amino acid sequence represented by SEQ ID NO: 2, and having a cyclization activity equivalent to or higher than that of SurE. The identity of amino acid sequences can be determined by use of a known search tool, such as FASTA and BLAST.

When the PBP-type TE of the present invention has a variant amino acid sequence of the amino acid sequence represented by SEQ ID NO: 2, it is preferable that in the variant amino acid sequence the amino acid sequence of the portion corresponding to the amino acid residues of 63 to 66 positions of SEQ ID NO: 2 is Ser-X1-X2-Lys, and/or the amino acid sequence of the portion corresponding to the amino acid residues of 153 to 158 positions of SEQ ID NO: 2 is Ser-Tyr-Ser-Asn-X3-Gly, and/or the amino acid sequence of the portion corresponding to the amino acid residues of 304 to 307 positions of SEQ ID NO: 2 is Gly-His-X4-Gly, and/or the amino acid sequence of the portion corresponding to the amino acid residues of 374 to 379 positions of SEQ ID NO: 2 is Gly-X5-X6-X7-Asn-Gly. The amino acid sequence of the portion corresponding to the amino acid residues of 374 to 379 positions of SEQ ID NO: 2 is more preferably Gly-X5-X6-X7-Asn-Gly. Note that, X1 to X7 each independently represent any amino acid residues. The corresponding portion is not necessarily a portion having the same amino acid residue numbers as in SEQ ID NO: 2 and may be a portion in the vicinity thereof. The corresponding portion can be found by comparing the amino acid sequence of the SurE mutant with SEQ ID NO: 2. Such comparison can be made by alignment using a known program such as BLAST or ClustalW.

Specific embodiments of the SurE mutant further include, but are not limited to, enzymes having an amino acid sequence that has a substitution, deletion, insertion or addition of one, several or several tens of amino acids in the amino acid sequence represented by SEQ ID NO: 2, and having a cyclization activity equivalent to or higher than that of SurE. The “several” refers to 2, 3, 5, 4, 6, 7, 8 or 9. The “several tens” refers to about 10 to about 90, for example, it may be about 20, about 30, about 40, about 50, about 60, about 70, about 80 or about 90, or the number between these numerical values. The substitution of an amino acid in an amino acid sequence may be a substitution with any amino acid, and preferably with an amino acid having a similar nature and/or structure (conservative amino acid substitution). For example, the amino acids within the parentheses: (G, A), (K, R, H), (D, E), (N, Q), (S, T, Y), (C, M), (F, W, Y, H), (V, L, I) may be mutually substituted.

Specific embosiments of the SurE mutant further include, but are not limited to, enzymes having an amino acid sequence encoded by a base sequence that hybridizes with a base sequence complementary to the base sequence represented by SEQ ID NO: 1 under stringent conditions, and having a cyclization activity equivalent to or higher than that of SurE.

The stringent conditions are known to those skilled in the art and refer to, for example, the following conditions:
Conditions where hybridization is performed in a buffer containing 0.25 M Na₂HPO₄, PH7.2, 7% SDS, 1 mM EDTA and 1×Denhardt's solution at a temperature of 60 to 68° C., preferably 65° C., and further preferably 68° C. for 16 to 24 hours, and thereafter, washing is performed twice in a buffer containing 20 mM Na₂HPO₄, pH7.2, 1% SDS and 1 mM EDTA at a temperature of 60 to 68° C., preferably 65° C., and further preferably 68° C. for 15 minutes; or
Conditions where prehybridization is performed in a hybridization solution containing 25% formamide, more stringently 50% formamide, 4×SSC (sodium chloride/sodium citrate), 50 mM HEPES pH7.0, 10×Denhardt's solution and 20 μg/ml denatured salmon sperm DNA at 42° C. overnight, and thereafter, washing is performed in a buffer containing 1×SSC and 0.1% SDS at 37° C., more stringently, in a buffer containing 0.5×SSC and 0.1% SDS at 42° C., and still more stringently in a buffer containing 0.2× Ssc and 0.1% SDS at 65° C.

The stringent conditions are not limited to the above examples.

The SurE mutant may be a naturally occurring one or artificially created by use of, for example, a genetic engineering method. In the case of a naturally occurring SurE mutant, the SurE mutant may be derived from any bacterium and may be a PBP-type TE that is possessed by an actinomycete other than Streptomyces albidoflavus NBRC 12854.

SurE and a mutant thereof can be obtained by a known method. For example, SurE and a mutant thereof can be produced by cloning a SurE gene, or a homologue or ortholog thereof by use of PCR, ligating the clone to an expression vector, introducing the expression vector into a host cell, and culturing the host cell. A recombinant SurE or a mutant thereof may be obtained by, for example, use of a method described in Examples of the specification. Alternatively, for example, a SurE mutant may be produced by use of a modified gene obtained by modifying the base sequence of a gene encoding SurE by use of a known method such as a site-specific mutagenesis.

Another preferable enzyme among the PBP-type TEs is WolJ (wollamide cyclase). WolJ is an enzyme PBP-type TE that is possessed by Streptomyces sp. MST-110588.

The base sequence of DNA encoding WolJ is represented by SEQ ID NO: 13. The amino acid sequence of WolJ is represented by SEQ ID NO: 14 (NCBI Reference Sequence: WP_242583930.1). In the present invention, an enzyme having the amino acid sequence represented by SEQ ID NO: 14 or an enzyme having the amino acid sequence encoded by the base sequence represented by SEQ ID NO: 13 is preferably used. The amino acid sequence of WolJ has an identity of 40.7% to the amino acid sequence of SurE.

A WolJ mutant may be used in the present invention. The WolJ mutant will be explained below. In the specification, unless otherwise specified, the term “WolJ” includes a mutant thereof.

A WolJ mutant having a cyclization activity equivalent to or higher than that of WolJ is preferably used in the present invention. The cyclization activity equivalent to or higher than that of WolJ refers to a cyclization activity of about 50% or more, preferably about 70% or more, more preferably about 80% or more, and still more preferably about 90% or more of the activity of WolJ. The cyclization activity of an enzyme can be measured by reacting a substrate and the enzyme and analyzing a product thereof. For example, cyclization activity may be measured by reacting a substrate and an enzyme, subjecting the resultant reaction mixture to LC-MS analysis and measuring the amount of a cyclized product, with reference to the procedure described in Examples of the specification.

Specific embodiments of the WolJ mutant include, but are not limited to, enzymes having an amino acid sequence having an identity of about 30% or more, for example about 35% or more, about 38% or more, preferably about 50% or more, more preferably about 70% or more (for example, 75% or more, 80% or more, and 85% or more), and still more preferably about 90% or more (for example, 91% or more, 92% or more, 93% or more, 94% or more, 95% or more, 96% or more, 97% or more, 98% or more, 99% or more, and 99.5% or more) to the amino acid sequence represented by SEQ ID NO: 14, and having a cyclization activity equivalent to or higher than that of WolJ. The identity of amino acid sequences can be determined by use of a known search tool such as BLASTP.

When the PBP-type TE of the present invention has a variant amino acid sequence of the amino acid sequence represented by SEQ ID NO: 14, it is preferable that in the variant amino acid sequence the amino acid sequence of the portion corresponding to the amino acid residues of 64 to 67 positions of SEQ ID NO: 14 is Ser-X1-X2-Lys, and/or the amino acid sequence of the portion corresponding to the amino acid residues of 157 to 162 positions of SEQ ID NO: 14 is Ser-Tyr-Ser-Asn-X3-Gly, and/or the amino acid sequence of the portion corresponding to the amino acid residues of 298 to 301 positions of SEQ ID NO: 14 is Gly-His-X4-Gly, and/or the amino acid sequence of the portion corresponding to the amino acid residues of 374 to 379 positions of SEQ ID NO: 14 is Gly-X5-X6-X7-Asn-Gly. It is more preferable that the amino acid sequence of the portion corresponding to the amino acid residues of 379 to 384 positions of SEQ ID NO: 14 is Gly-X5-X6-X7-Asn-Gly. Note that, X1 to X7 each independently represent any amino acid residues. The corresponding portion is not necessarily a portion having the same amino acid residue numbers as in SEQ ID NO: 14 and may be a portion in the vicinity thereof. The corresponding portion can be found by comparing the amino acid sequence of the SurE mutant with SEQ ID NO: 14. Such comparison can be made by alignment using a known program such as BLAST or ClustalW.

Specific embodiments of the WolJ mutant further include, but are not limited to, enzymes having an amino acid sequence that has a substitution, deletion, insertion or addition of one, several or several tens of amino acids in the amino acid sequence represented by SEQ ID NO: 14, and having a cyclization activity equivalent to or higher than that of WolJ. The “several” refers to 2, 3, 5, 4, 6, 7, 8 or 9. The “several tens” refers to about 10 to about 90, for example, it may be about 20, about 30, about 40, about 50, about 60, about 70, about 80 or about 90, or the number between these numerical values. The substitution of an amino acid in an amino acid sequence may be a substitution with any amino acid, and preferably with an amino acid having a similar nature and/or structure (conservative amino acid substitution). For example, the amino acids within the parentheses: (G, A), (K, R, H), (D, E), (N, Q), (S, T, Y), (C, M), (F, W, Y, H), (V, L, I) may be mutually substituted.

Specific embodiments of the WolJ mutant further include, but are not limited to, enzymes having an amino acid sequence encoded by a base sequence that hybridizes with a base sequence complementary to the base sequence represented by SEQ ID NO: 13 under stringent conditions, and having a cyclization activity equivalent to or higher than that of WolJ.

The stringent conditions are the same as defined above.

The WolJ mutant may be a naturally occurring one or artificially created by use of, for example, a genetic engineering method. In the case of a naturally occurring WolJ mutant, the WolJ mutant may be derived from any bacterium and may be derived from an actinomycete other than Streptomyces sp. MST-110588.

WolJ and a mutant thereof can be obtained by a known method. For example, WolJ and a mutant thereof can be produced by cloning a WolJ gene, or a homologue or ortholog thereof by use of PCR, ligating the clone to an expression vector, introducing the expression vector into a host cell, and culturing the host cell. A recombinant WolJ and a mutant thereof may be obtained by, for example, use of a method described in Examples of the specification. Alternatively, for example, a WolJ mutant may be produced by use of a modified gene obtained by modifying the base sequence of a gene encoding WolJ by use of a known method such as a site-specific mutagenesis.

In another embodiment of the above aspect, the catalyst is a TycC-TE. The TycC-TE is an enzyme domain that catalyzes a peptide cyclization reaction, positioned at the C terminal of a non-ribosomal peptide synthetase TycC. The TycC-TE is characterized by catalyzing a cyclization reaction of a short linear peptide having about 15 residues or less, for example, about 10 residues, and exhibiting a broad substrate selectivity (for TycC-TE, see, for example, “Peptide cyclization catalysed by the thioesterase domain of tyrocidine synthetase”; Trauger J W, Kohli R M, Mootz H D, Marahiel M A, Walsh C T. Nature. 2000 Sep. 14; 407 (6801): 215-8. doi: 10.1038/35025116. PMID: 11001063, and “Biomimetic synthesis and optimization of cyclic peptide antibiotics”; Kohli R M, Walsh C T, Burkart M D. Nature. 2002 Aug. 8; 418 (6898): 658-61. doi: 10.1038/nature 00907. PMID: 12167866).

In the present invention, the TycC-TE is an enzyme that can cyclize a peptide substrate having a diol as a leaving group attached to the carboxyl group of the C-terminal residue, in a head-to-tail mode. An organism from which the TycC-TE to be used in the present invention is derived is not particularly limited, and is preferably a bacterium, for example, the organism may be a bacterium of the genus Brevibacillus. Preferred examples of the TycC-TE include those derived from Brevibacillus parabrevis ATCC 8185. The TycC-TE may be derived from a bacterium other than the above bacterium. The TycC-TE can be obtained by using a known cloning method.

The base sequence of DNA encoding a TycC-TE derived from Brevibacillus parabrevis ATCC 8185 is represented by SEQ ID NO: 6. The amino acid sequence of TycC-TE derived from Brevibacillus parabrevis ATCC 8185 is represented by SEQ ID NO: 7. In the present invention, an enzyme having the amino acid sequence represented by SEQ ID NO: 7 or an enzyme having the amino acid sequence encoded by the base sequence represented by SEQ ID NO: 6 is preferably used.

A TycC-TE mutant may be used in the present invention. The TycC-TE mutant will be explained below. In the specification, unless otherwise specified, the term “TycC-TE” includes its mutants.

A TycC-TE mutant having a cyclization activity equivalent to or higher than that of TycC-TE is preferably used in the present invention. The cyclization activity equivalent to or higher than that of TycC-TE refers to a cyclization activity of about 50% or more, preferably about 70% or more, more preferably about 80% or more, and still more preferably about 90% or more of the cyclization activity of TycC-TE. The cyclization activity of an enzyme can be measured by reacting a substrate and the enzyme and analyzing a product thereof. For example, cyclization activity may be measured by reacting a substrate and an enzyme, subjecting the resultant reaction mixture to LC-MS analysis and measuring the amount of a cyclized product, with reference to the procedure described in Examples of the specification.

Specific embodiments of the TycC-TE mutant include, but are not limited to, enzymes having an amino acid sequence having an identity of about 30% or more, for example about 35% or more, about 38% or more, preferably about 50% or more, more preferably about 70% or more (for example, 75% or more, 80% or more, and 85% or more), and still more preferably about 90% or more (for example, 91% or more, 92% or more, 93% or more, 94% or more, 95% or more, 96% or more, 97% or more, 98% or more, 99% or more, and 99.5% or more) to the amino acid sequence represented by SEQ ID NO: 7, and having a cyclization activity equivalent to or higher than that of TycC-TE. The identity of amino acid sequences can be determined by use of a known search tool, such as BLASTP.

When the TycC-TE of the present invention has a variant amino acid sequence of the amino acid sequence represented by SEQ ID NO: 7, it is preferable that the amino acid residues in the variant amino acid sequence corresponding to the amino acid residues, serine at position 82, aspartic acid at position 109 and histidine at position 226, counted from the N terminal of the amino acid sequence represented by SEQ ID NO: 7 are identical to these, or are amino acid residues homologous to the same family or amino acid residues satisfying conservative amino acid substitutions. Homologous amino acids and conservative amino acid substitutions are known to those skilled in the art. Such a variant amino acid sequence has an amino acid sequence identity as mentioned above to the amino acid sequence represented by SEQ ID NO: 7. The TycC-TE mutant of the present invention is not limited to those explained above. The “corresponding amino acid residue” in the amino acid sequence of an enzyme mutant can be found in consideration of, e.g., the position counted from the N terminal, and the amino acid sequence in the vicinity of the position and computational structure prediction.

Specific embodiments of the TycC-TE mutant further include, but are not limited to, enzymes having an amino acid sequence that has a substitution, deletion, insertion or addition of one, several or several tens of amino acids in the amino acid sequence represented by SEQ ID NO: 7, and having a cyclization activity equivalent to or higher than that of TycC-TE. The “several” refers to 2, 3, 5, 4, 6, 7, 8 or 9. The “several tens” refer to about 10 to about 90, for example, it may be about 20, about 30, about 40, about 50, about 60, about 70, about 80 or about 90, or the number between these numerical values. The substitution of an amino acid in an amino acid sequence may be a substitution with any amino acid, and preferably with an amino acid having a similar nature and/or structure (conservative amino acid substitution). For example, the amino acids within the parentheses: (G, A), (K, R, H), (D, E), (N, Q), (S, T, Y), (C, M), (F, W, Y, H), (V, L, I) may be mutually substituted.

Specific embodiments of the TycC-TE mutant further include, but are not limited to, enzymes having an amino acid sequence encoded by a base sequence that hybridizes with a base sequence complementary to the base sequence represented by SEQ ID NO: 6 under stringent conditions, and having a cyclization activity equivalent to or higher than that of TycC-TE.

The stringent conditions are the same as defined above.

The TycC-TE mutant may be a naturally occurring one or artificially created by use of, for example, a genetic engineering method. For example, the TycC-TE mutant may be derived from a bacterium of the phylum Firmicutes.

A TycC-TE and a mutant thereof can be obtained by a known method. For example, the TycC-TE and a mutant thereof can be produced by cloning a TycC-TE gene, or a homologue or ortholog thereof by use of PCR, ligating the clone to an expression vector, introducing the expression vector into a host cell and culturing the host cell. A recombinant TycC-TE and a mutant thereof may be obtained by, for example, use of a method described in Examples of the specification. Alternatively, for example, a TycC-TE mutant may be produced by use of a modified gene obtained by modifying the base sequence of a gene encoding a TycC-TE by use of a known method such as a site-specific mutagenesis.

In the specification, a peptide is a molecule having amino acid residues linked via a peptide bond. The number of amino acid residues is 2 or more. In the specification, a peptide includes an oligopeptide, a polypeptide, and a protein. In the specification, all linkages between amino acid residues in a peptide are not necessarily peptide bonds. Amino acids constituting a peptide may or may not be included in naturally occurring proteins (for example, amino acids other than α-amino acids, such as β-alanine and γ-aminobutyric acid, ornithine, homocysteine). Amino acids constituting a peptide may be L-form or D-form amino acids. Amino acids constituting a peptide may be present in living organisms or artificially synthesized. Amino acids constituting a peptide may be modified. For example, a side-chain carboxyl group may be esterified; hydrogen of a side-chain amino group may be substituted with an alkyl group; a side-chain SH group and another molecule may form an S—S bond; a ring such as a side-chain phenyl group may be substituted with, e.g., OH, halogen, an alkyl group. Alternatively, a glycoside may be formed via a side-chain OH group. The peptide may contain a non-peptidic structure (described later). In the specification, the left end of a peptide is the N-terminal and the right end is the carboxyl-terminal. In the specification, the position of an amino acid residue in a peptide is defined by the number counted from the N-terminal. In the specification, the nomenclature of amino acids and amino acid residues follows one-letter codes or three-letter codes known in the technical field.

The substrate to be used in the cyclization reaction of the present invention is a peptide. The composition and length of the substrate to be used in the present invention are not particularly limited. The substrate is usually a linear peptide, however it may be a peptide having a branch structure or a peptide partially having a ring structure. The substrate may be a peptide in which a peptide bond is cleaved in the cyclic peptide to be obtained. The substrate, if linear, may have a length of several amino acids or more. The upper limit of the length of the substrate is not particularly limited, and it may be, for example, about 15 amino-acid length or less. Specific examples of the length of the substrate include 4 amino acid-length, 5 amino acid-length, 6 amino acid-length, 7 amino acid-length, 8 amino acid-length, 9 amino acid-length, 10 amino acid-length, 11 amino acid-length, 12 amino acid-length, 13 amino acid-length, 14 amino acid-length and 15 amino acid-length or more. In the case where a PBP-type TE such as SurE is used, it is preferable that the C-terminal amino acid of a substrate is a D-amino acid and the N-terminal amino acid thereof is an L-amino acid. When a PBP-type TE is used, a cyclization reaction takes place even if a substrate has a bulky amino acid residue and/or hydrophobic amino acid residue at the C terminal and/or N terminal. Accordingly, if a PBP-type TE is used, condensation of bulky amino acid residues, which is difficult to be made by chemical synthesis, can be made. In contrast, a SurE mutant (G235L) can cyclize a substrate even if it has a small amino acid such as glycine at the C terminal. When a TycC-TE is used, it is preferable that the C-terminal amino acid of a substrate is an L-amino acid and the N-terminal amino acid thereof is a D-amino acid. When a TycC-TE is used, the N-terminal amino acid of a substrate is preferably D-phenylalanine. When WolJ is used, the C-terminal amino acid of a substrate is preferably D-Arg, D-Orn or Gly. The substrates for SurE, TycC-TE, WolJ and mutants thereof are not limited to those mentioned above. As described above, various substrate peptides can be cyclized by varying the type of enzyme to be used.

It is preferable to activate the carboxyl group of the C-terminal amino acid of a substrate. The carboxyl group may be activated by adding a leaving group. Examples of the leaving group include, but are not limited to, an alcohol, a phenol and a thiol. A preferable leaving group is, for example, an alcohol and, in particular, a diol. A typical example of the diol is an alkyl group having two hydroxyl groups. EG or an EG analogue is a more preferable leaving group and EG is a further preferable leaving group. Examples of the EG analogue include, but are not limited to, diols having 1 to 4 carbon atoms such as methylene glycol, propylene glycol and 1, 3-butanediol.

The substrate may have a non-peptidic structure. The non-peptidic structure refers to a structure that a natural peptide does not have. The type and the configuration thereof are not particularly limited. Examples of the non-peptidic structure include, but are not limited to, marker substances such as biotin, fluorescein, rhodamine and luciferin, sugars, lipids, bases, spacers such as —(CH₂)ₙ— (n is an integer of 1 or more), several glycine residues (Gly)ₘ(m is an integer of 1 or more), and PEG (for example, the number of polymerized molecules: 1 to 9). The non-peptidic structure may be naturally occurring one or artificially created. The non-peptidic structure may be bound to a side chain of the amino acids constituting a substrate or inserted between amino acids constituting a substrate. A polyketide skeleton or a peptide nucleic acid may be present in part of a substrate. Preferably, the non-peptidic structure is bound to a substrate in such a manner that the catalytic action of a PBP-type TE or a TycC-TE is not prevented.

In another aspect, the present invention provides a peptide having a diol attached to the C-terminal amino acid. The peptide is subjected to a cyclization reaction using a PBP-type TE or a TycC-TE. In this case, a diol acts as a leaving group.

It is preferable that a substrate to be used in a method for producing a cyclic peptide of the present invention is obtained by a synthesis method including the following steps:
(i) elongating a peptide from a diol carried on a solid phase, and
(ii) cleaving the peptide to which the diol is bound from the solid phase.

The solid phase used in the step (i) of the method for producing a substrate as mentioned above, may be, for example, a resin. Any resin may be used as long as it can carry a diol serving as a leaving group. The resin may be, for example, a chlorotrityl resin. Binding of a diol to a solid phase can be appropriately performed by those skilled in the art depending on the types of resin and diol. To a diol carried on a solid phase in advance, individual amino acids (side chains may be appropriately protected) constituting a desired peptide are sequentially connected (peptide chain elongation) to obtain the desired peptide bound on the solid phase via the diol. The peptide chain elongation can be performed in accordance with a known method, for example, the Fmoc method. As the diol, EG or an analogue thereof is preferable and EG is more preferable.

Then, in the step (ii), the desired peptide is cleaved from the solid phase to obtain a peptide having a diol attached at the C terminal. Before or after the cleavage or simultaneously with the cleavage, a side-chain protecting group can be removed. Preferably, a side-chain protecting group is removed simultaneously with the cleavage. The cleavage and deprotection can be performed by use of a known method.

In the above method, since solid phase synthesis of a peptide having a leaving group attached thereto is conducted, a condensation step of a leaving group in a liquid phase can be avoided. Owing to the avoidance of the condensation step of a leaving group, the production of isomers that are difficult to be separated, and the resulting reduction in yield can be avoided. In addition, a purification step by e.g., high-performance liquid chromatography and essential for conventional substrate synthesis routes can be skipped. As a whole, significant improvement of the yield and simplification can be realized. Since EG is extremely inexpensive, the production cost of a substrate is significantly reduced by using EG as a leaving group. Accordingly, the method for synthesizing a cyclic peptide according to the present invention can be performed at a low cost. The method for producing a substrate can be expected to greatly contribute to the establishment of a synthesis/mass synthesis method for a green and simple cyclic peptide library.

In a further aspect, the present invention provides a kit comprising a PBP-type TE or a TycC-TE, for peptide cyclization. Usually, the kit is attached with an instruction or a handling manual. The kit may further contain a substrate having a diol as a leaving group attached to the carboxyl group of the C-terminal residue or a means for producing the substrate. Examples of the means for producing the substrate include, but are not limited to, a resin that can carry a diol, a resin on which a diol is carried, a reagent for adding a diol to a resin, and amino acids protected for solid-phase synthesis.

The terms in the specification, unless otherwise specified, are interpreted as ordinarily understood in the fields of, e.g., chemistry, biology, biochemistry, medicine and pharmacy. In the specification, “about” before a numerical value represents the numerical value±20%, preferably the numerical value±10%, and more preferably the numerical value±5%.

The present invention will be more specifically described by way of Examples below. Examples are however just provided for explanation and should not be construed as limiting the scope of the present invention.

### Example 1

### Cyclization Reaction by SurE

### 1. Synthesis of Substrate

### (1) Protocol for Solid-Phase Peptide Synthesis

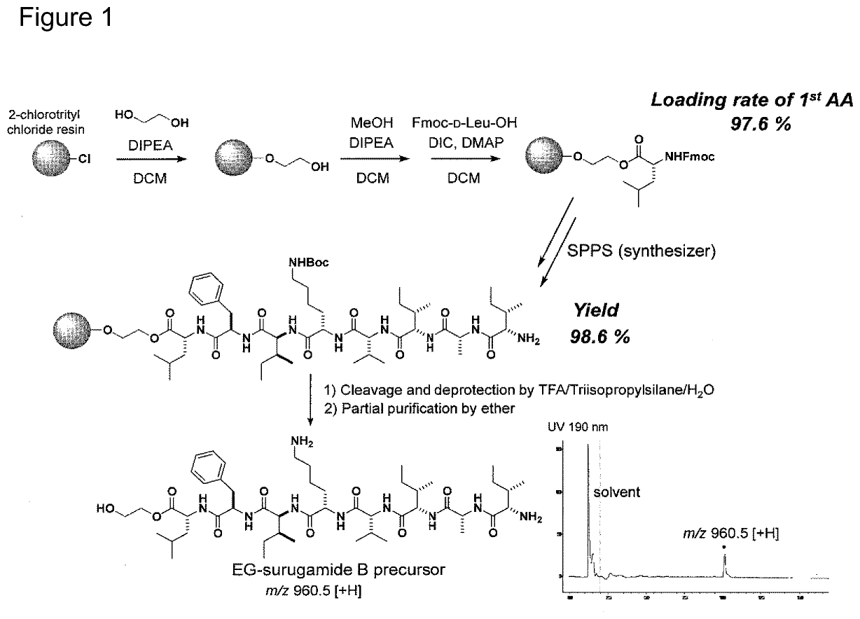

A substrate was synthesized by a solid-phase peptide synthesis method. After ethylene glycol was condensed with a chlorotrityl resin, a peptide elongation reaction was repeatedly performed with DIC/Oxyma. The substrate peptide was cleaved from the resin simultaneously with removal of a side-chain protecting group, with TFA. The obtained substrate was purified by a precipitation method with ether. See FIG. 1.

The solid-phase peptide synthesis protocol used for the substrate synthesis included the following steps 1 to 4.

Step 1: The Fmoc group of a peptide supported on a solid phase was removed by using a 20% piperidine/DMF solution (10 minutes, room temperature).

Step 2: The resin in the reaction vessel was washed with DMF (×3) and CH₂Cl₂ (×3).

Step 3: To a solution of building blocks (4 eq) protected by F-moc, DIC (4 eq) and Oxyma (4 eq in DMF) in NMP were added. Two to three minutes after pre-activation, the mixture was poured in a reaction vessel. The resultant mixture was stirred for 30 minutes.

Step 4: The resin in the reaction vessel was washed with DMF (×3) and CH₂Cl₂ (×3).

Steps 1 to 4 were repeated to concentrate amino acids on the solid support.

### (2) Synthesis of Substrate-EG Conjugate 1

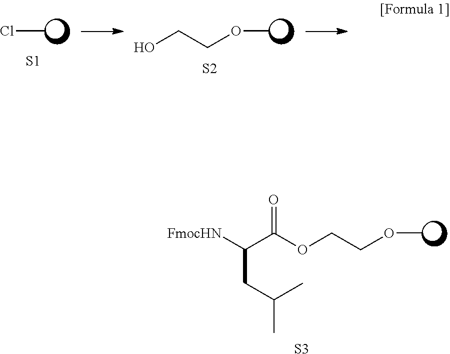

2-Chloroethyl resin S1 (A00187, manufactured by Watanabe Chemical Industries, Ltd., 120 mg, 0.188 mmol) in a LibraTube (registered trademark) was swollen with CH₂Cl₂ for 10 minutes, and subsequently, the excess solvent was removed by filtration. To the resultant resin, a solution of ethylene glycol (42.1 mg, 0.752 mmol) and i-Pr₂NEt (163.7 μL, 0.940 mmol) in CH₂Cl₂ (2.0 mL) was added. The resultant mixture was stirred at 37° C. for 2 hours to obtain ethylene glycol-2-chlorotrityl resin S2. To resin S2, a solution of Fmoc-D-Leu-OH (132.9 mg, 0.376 mmol), DIC (117.7 μL, 0.752 mmol) and DMAP (0.00188 mmol, 0.230 mg) in CH₂Cl₂ (2.0 mL) was added. The resultant mixture was stirred at 37° C. for 3 hours to obtain Fmoc-D-Leu-ethylene glycol-2-chlorotrityl resin S3. To resin S3 (7.8 mg) dried, 20% piperidine in DMF was added and the resultant mixture was stirred for one hour. The supernatant was diluted with DMF and ultraviolet absorbance was measured at 301 nm. Based on the absorbance (0.3675) obtained, a loading rate was determined as 0.604 mmolg⁻¹.

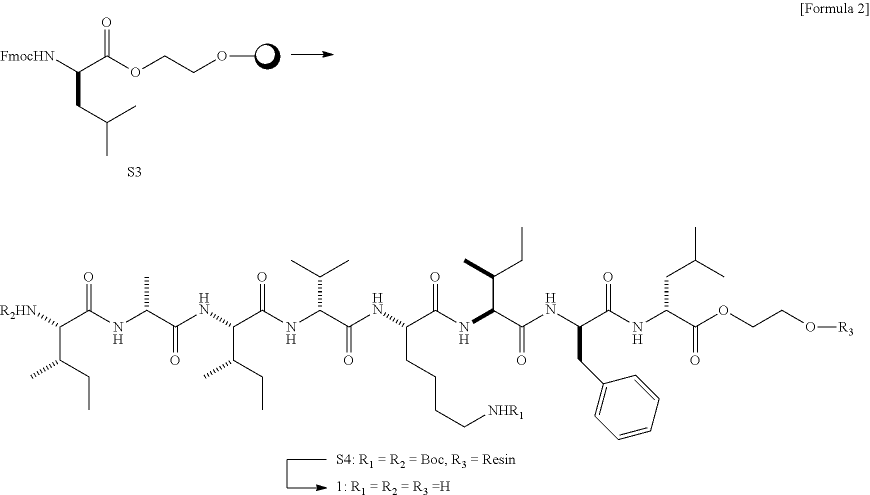

Resin S3 (0.05 mmol, loading rate 0.604 mmol/g, 82.8 mg) in LibraTube (registered trademark) was swollen with DMF for 10 minutes, and subsequently, subjected to solid-phase peptide synthesis consisting of 7 cycles [Fmoc-D-Phe-OH, Fmoc-L-Ile-OH, Fmoc-L-Lys (Boc)-OH, Fmoc-D-Val-OH, Fmoc-L-Ile-OH, Fmoc-D-Ala-OH, Boc-L-Ile-OH]. The synthesis was performed in accordance with the protocol (the above Steps 1 to 4) to obtain resin-bound peptide S4. To peptide S4, TFA/H₂O/iPr₃SiH=95:2.5:2.5 (1.0 mL) was added while stirring, and subsequently, the reaction mixture was filtered. The reaction was repeated 6 times. The filtrate was diluted with Et₂O (12 mL), cooled (−30° C.) and then centrifuged at 3500×g and 4° C. for 10 minutes to obtain crude peptide 1 (EG-surugamide B precursor in FIG. 1) (43.1 mg, 0.0449 mmol, yield: about 89.8%). In an LC-MS chart of crude peptide 1, a single peak appeared (FIG. 1, m/z 960.5 [+H]). Crude peptide 1 was used as a substrate for a cyclization reaction.

### 2. Production of Recombinant SurE

### (1) Construction of Plasmid

A DNA fragment (GeneBank protein ID BBZ90014.1) encoding SurE was amplified from the genomic DNA of Streptomyces albidoflavus NBRC12854 by use of polymerase KOD One (TOYOBO) and the following an oligonucleotide primer set (SEQ ID NO: 3 in which an EcoRI recognition site was underlined, SEQ ID NO: 4 in which a HindIII recognition site was underlined).

Forward primer:
(SEQ ID NO: 3)
5′-CCGGAATTCCATATGGGTGCCGAGGGGGCG-3′
Reverse primer:
(SEQ ID NO: 4)
5′-CCCAAGCTTTCAGAGCCGGTGCATGGC-3′

The fragment amplified was inserted into the multi-cloning site of pET-28a (+) (Novagen) to obtain an expression vector (SurE-pET28a) for preparing recombinant SurE.

### (2) Preparation of Recombinant SurE

The expression vector obtained above was introduced in E. coli BL21 (DE3). A single colony was inoculated in 10 mL of 2×YT medium (1.6% Bacto tryptone, 1.0% Bacto Yeast extract, 0.5% NaCl) containing 50 μg/mL kanamycin, cultured at 37° C. overnight and used as a seed culture. A culture (2.0 mL) was transferred to 200 mL of 2×YT medium containing 50 μg/mL kanamycin and cultured at 37° C. for 3 hours. The resultant culture was cooled on ice and 0.1 mM IPTG was added to induce the expression of recombinant SurE. E. coli was cultured at 16° C. overnight. Cells were collected by centrifugation (3500×g, 10 minutes) and ground by an ultrasonic homogenizer. After the residue was removed by centrifugation (17,000×g, 10 minutes), a fraction containing a soluble protein was subjected to Ni-NTA affinity column (Merck Millipore) equilibrated with a wash buffer (20 mM Tris-HCl pH8.0, 150 mM NaCl, 20 mM imidazole). The column was washed with the wash buffer and elution was performed with a 500 mM imidazole wash buffer. A column eluate was concentrated by an Amicon Ultra 0.5 mL filter (Merck Millipore). The concentration of a protein solution was measured by a Bio-Rad protein assay kit. Note that, in this experiment, recombinant SurE was obtained as a protein having a histidine tag attached to the N-terminal side. The amino acid sequence is represented by SEQ ID NO: 5.

### 3. Cyclization Reaction by Recombinant SurE

### (1) Experimental Method

A reaction mixture (100 μL in volume) containing 20 mM Tris-HCl (pH7.7) and 200 μM crude peptide 1 was prepared. A reaction was initiated by adding 4 μM SurE to the reaction mixture. After addition of the enzyme, the reaction mixture was incubated at 30° C. for 3 hours and the reaction was terminated by adding 0.1% TFA of the same volume thereto. The same volume of methanol was added to further dilute the sample. The diluted sample was centrifuged at 20,000×g for 10 minutes. The supernatant obtained was analyzed by LC-MS (amazon SL-NPC), which was used in combination with the Shimadzu HPLC system and driven in a positive mode. Separation was performed by use of Cosmosil 5C18-MS-II 2.0×150 mm column (Nacalai Tesque). H₂O+0.05% TFA and acetonitrile+0.05% TFA were used as mobile phases A and B, respectively. Elution of the sample was performed at a flow rate of 0.2 ml·min⁻¹ and in a gradient mode (the ratio of mobile phase B was changed from 10% to 90% over 20 minutes).

### (2) Experimental Results

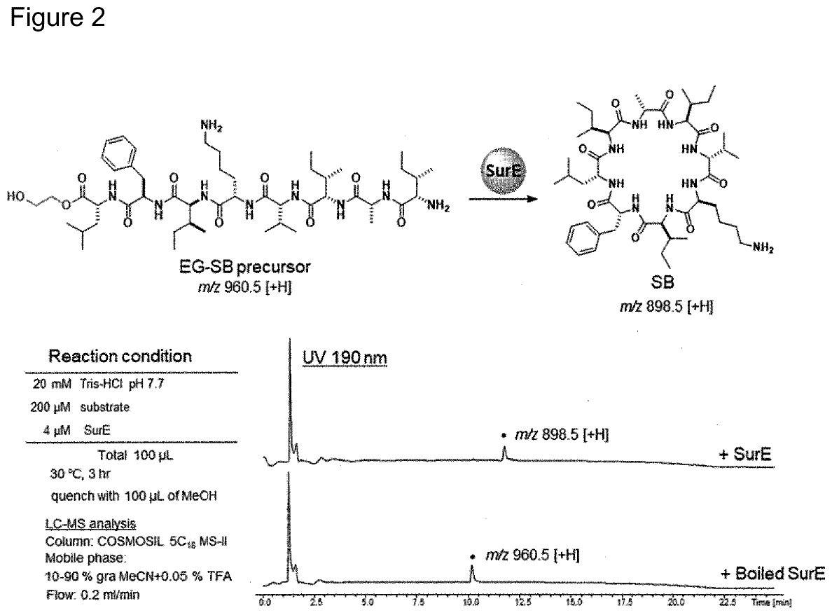

The results of cyclic peptide enzymatic synthesis are shown in FIG. 2. The upper LC-MS chart shows the analysis result of the reaction mixture having active SurE added thereto. The lower LC-MS chart shows the analysis result of the reaction mixture having inactivated SurE by boiling added thereto. It was confirmed that a substrate (crude peptide 1, EG-SB precursor, m/z 960.5 [+H]) disappeared by the action of SurE to generate a cyclic peptide (surugamide B (SB), m/z 898.5 [+H]).

### Example 2

### Cyclization Reaction by SurE Mutant

### 1. Synthesis of Substrate

In the same manner as in Example 1, 1 (1) and (2), seco-desprenylagaramide C-EG shown below was synthesized.

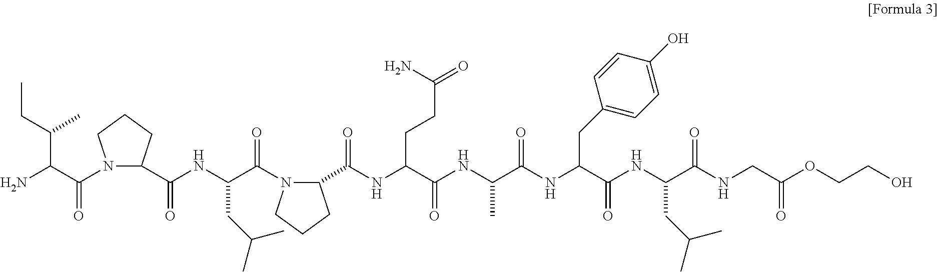 [SMILES: CC[C@H](C)C(N)C(=O)N1CCCC1C(=O)N[C@@H](CC(C)C)C(=O)N1CCC[C@H]1C(=O)NC(CCC(N)=O)C(=O)N[C@@H](C)C(=O)NC(Cc1ccc(O)cc1)C(=O)N[C@@H](CC(C)C)C(=O)NCC(=O)OCCO]

### 2. Production of SurE Mutant

### (1) Construction of Plasmid

Using the following primer set:

Forward primer:
(SEQ ID NO: 10)
5′-TCGCTGGCCGCCGCCCTGGGGCTGGTCGGCAG-3′
Reverse primer:
(SEQ ID NO: 11)
5′-CTGCCGACCAGCCCCAGGGCGGCGGCCAGCGA-3′

and using SurE-pET28a as a template, and a fragment was amplified by use of polymerase KOD One (TOYOBO), and introduced into E. coli DH5α to prepare a plasmid SurE (G235L)-pET28a for expressing a mutant.

### (2) Preparation of Recombinant SurE Mutant (SurE (G235L))

In the same manner as in Example 1, 2. (2), SurE (G235L) was obtained. The amino acid sequence thereof is represented by SEQ ID NO: 12. In SEQ ID NO: 12, glycine at position 255, counted from the N terminal (corresponding to glycine at position 235, counted from the N terminal of wild type SurE (SEQ ID NO: 2)) is substituted with leucine.

### 3. Cyclization Reaction by SurE (G235L)

### (1) Experimental Method

The experiment was performed in accordance with the method of Example 1, 3. (1). Using SurE (G235L) as an enzyme and seco-desprenylagaramide C-EG as a substrate, an enzymatic reaction was performed at 16° C. for 30 hours.

### (2) Experimental Results

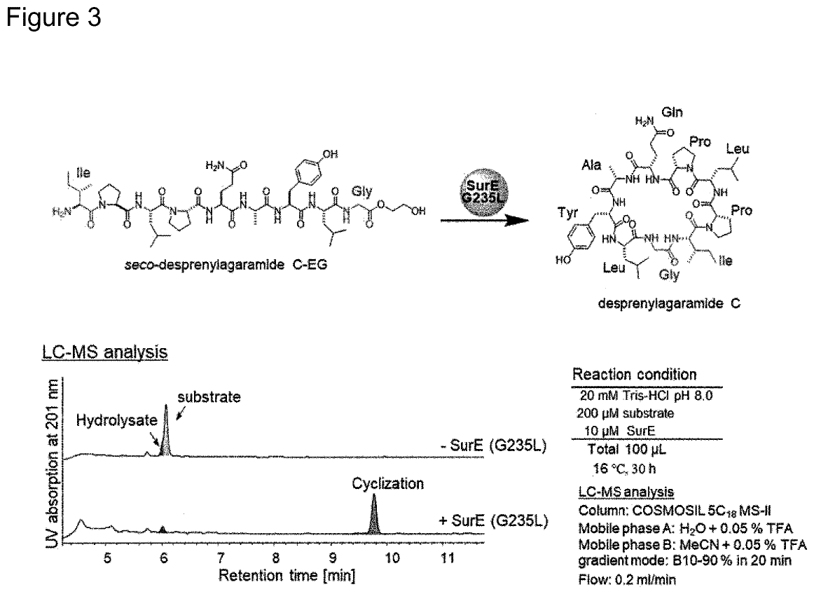

The results of the cyclic peptide enzymatic synthesis are shown in FIG. 3. In the figure, the upper LC-MS analysis chart shows the results of an enzyme-free system and the lower chart shows the results of an enzyme-containing system. It was confirmed that the substrate disappeared by an enzymatic reaction to generate a cyclized product (desprenylagaramide C). It was also found that a peptide having glycine at the C terminal, which cannot be cyclized by wild-type SurE, can be cyclized.

### Example 3

### Cyclization Reaction by WolJ

1. Synthesis of Substrate In the same manner as in Example 1, 1 (1) and (2), seco-wollamide B1-EG shown below was synthesized.

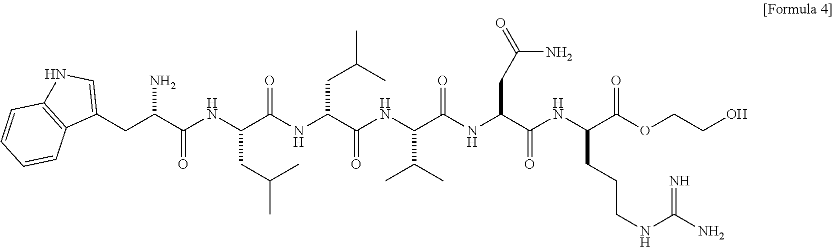 [SMILES: CC(C)C[C@H](NC(=O)[C@@H](N)Cc1c[nH]c2ccccc12)C(=O)N[C@H](CC(C)C)C(=O)N[C@H](C(=O)N[C@@H](CC(N)=O)C(=O)N[C@H](CCCNC(=N)N)C(=O)OCCO)C(C)C]

### 2. Production of Recombinant WolJ

DNA (SEQ ID NO: 15) was synthesized based on the gene sequence of WolJ (GenBank: UNO41476.1), in which codons were optimized for E. coli, and inserted into an NdeI/HindIII site of pCold II (TakaRa) to construct an expression plasmid for WolJ having a His tag at the N terminal. A method for preparing recombinant WolJ was the same as described in Example 1, 2. (2). The amino acid sequence of the obtained recombinant WolJ is represented by SEQ ID NO: 16.

### 3. Cyclization Reaction by Recombinant WolJ

### (1) Experimental Method

An experiment was performed in accordance with the method of Example 1, 3. (1). Using recombinant WolJ (obtained in accordance with the procedure described in the above section 2) as an enzyme and seco-wollamide B1-EG as a substrate, an enzymatic reaction was performed at 30° C. for 2 hours. The flow rate of an HPLC eluent was set at 0.4 ml/minute.

### (2) Experimental Results

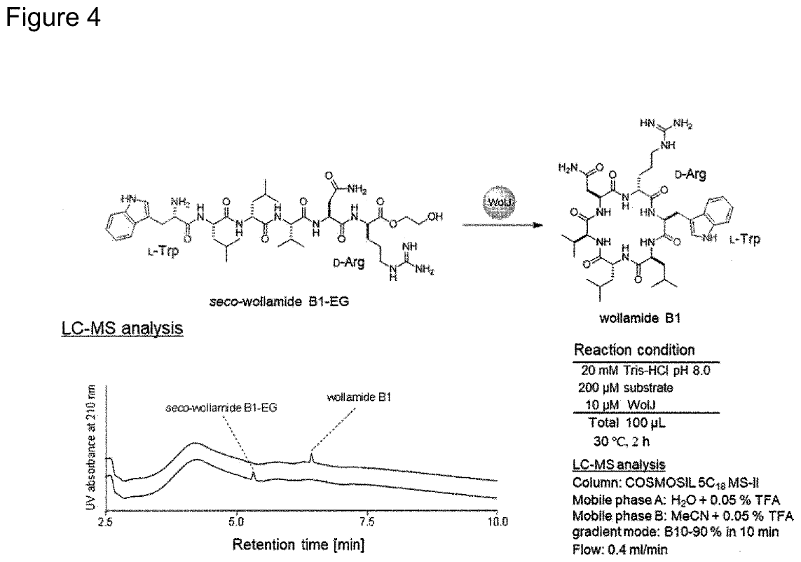

The results of the cyclic peptide enzymatic synthesis are shown in FIG. 4. In the figure, the upper LC-MS analysis chart shows the results of an enzyme-containing system and the lower chart shows the results of an enzyme-free system. It was confirmed that the substrate disappeared by an enzymatic reaction to generate a cyclized product (wollamide B1). It was also found that a peptide having D-Arg at the C terminal, which is a cause of a low cyclization efficiency in SurE, can be efficiently cyclized.

### Example 4

### Cyclization Reaction of a Peptide Having Polyethylene Glycol (PEG) Chain Therein by SurE

### 1. Synthesis of Substrate

Condensation of Fmoc-AEEA-OH with S3 synthesized by the method described in Example 1, 1. (2) was performed 1 to 7 times in accordance with a conventional method. Finally, L-Ile-OH was condensed, and then, cleavage from a resin and ether precipitation were performed in the same manner as in Example 1, 1. (2). The substrate synthesized (having EG at the C terminal) is shown below. The size of the peptide corresponds to the size of a peptide having 5 to 23 amino acid residues.

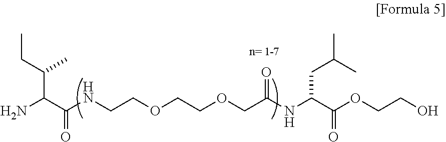

### 2. Enzyme

SurE prepared in Example 1 was used.

### 3. Cyclization Reaction

### (1) Experimental Method

An experiment was performed in accordance with the method of Example 1, 3. (1). Using a peptide having a polyethylene glycol (PEG) chain therein as a substrate, an enzymatic reaction was performed at 30° C. for 4 hours. Elution was performed by HPLC using a 37% acetonitrile+0.05% TFA in an isocratic mode.

### (2) Experimental Results

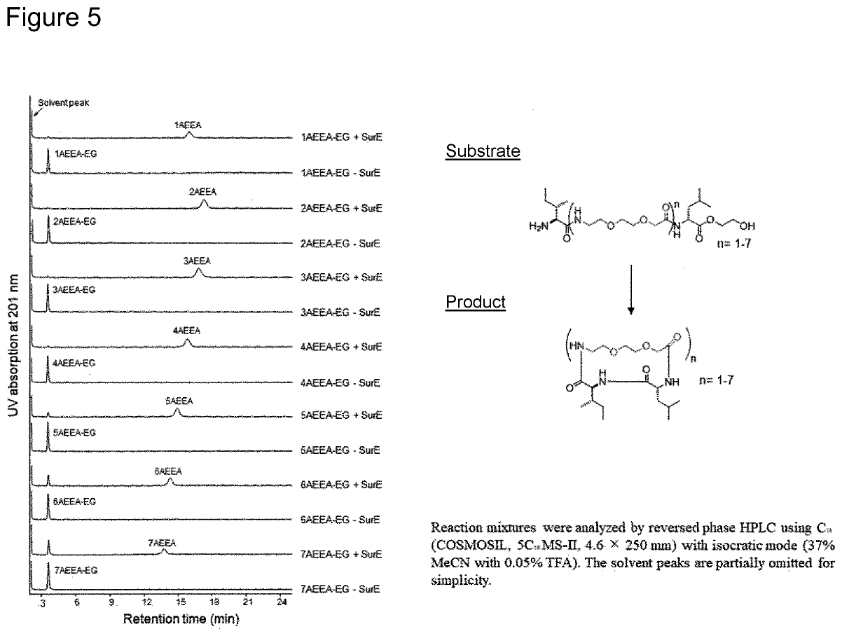

The results of the cyclic peptide enzymatic synthesis are shown in FIG. 5. As shown in the LC-MS analysis charts of FIG. 5, it was confirmed that SurE efficiently circularized also a peptide having a PEG chain therein.

### Example 5

### Cyclization Reaction by TycC-TE

### 1. Synthesis of Substrate

A substrate was synthesized in accordance with the method described in Example 1. The substrate is a precursor of EG-tyrocidine A shown below.

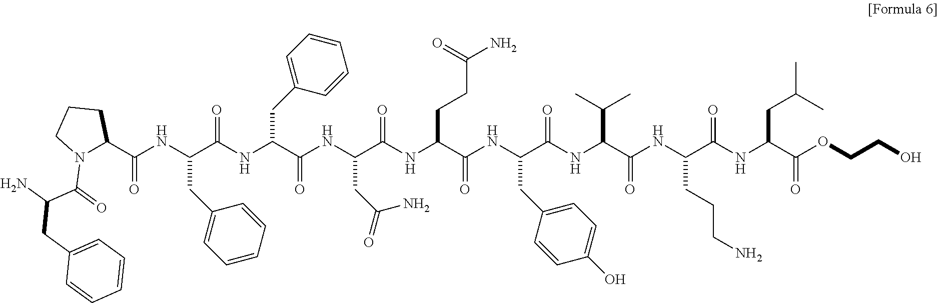

### 2. Production of Recombinant TycC-TE

A nucleic acid (codons were optimized for E. coli) encoding the amino acid sequence encoded by the region from the positions 6234 to 6486 of TycC (UniProtKB/Swiss-Prot: 030409.1) was synthesized. The base sequence synthesized is represented by SEQ ID NO: 8. The base sequence was inserted into an NdeI/HindIII site of a pET28a (+) to obtain a vector (TycCTE-pET28a) for expressing a recombinant TycCTE. In the same manner as in Example 1, the vector was introduced in E. coli. The E. coli was cultured to obtain recombinant TycC-TE. The amino acid sequence thereof is represented by SEQ ID NO: 9.

### 3. Cyclization Reaction by Recombinant TycC-TE

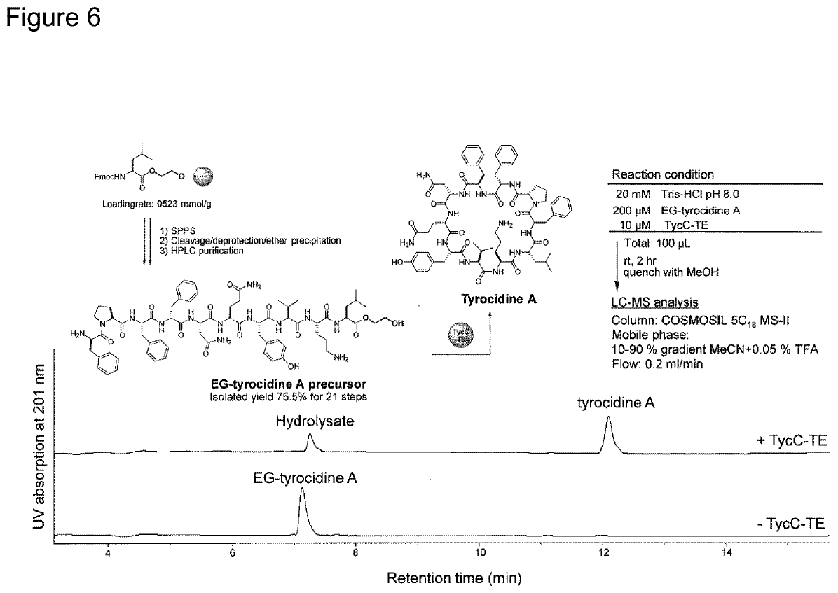

Synthesis and cyclization reaction of a substrate were performed in accordance with the method described in Example 1. The substrate synthesized was a tyrocidine A precursor (see FIG. 6) having EG as a leaving group and cyclase was TycC-TE.

A reaction product was analyzed by LC-MS. The results are shown in FIG. 6. In an active TycC-TE containing system, it was confirmed that a cyclic peptide, i.e., tyrocidine A, and a small amount of substrate hydrolysate were generated (+TycC-TE, FIG. 6).

### INDUSTRIAL APPLICABILITY

The present invention can be used for producing known and novel cyclic peptides. Thus, the present invention can be used for producing e.g., pharmaceuticals, physiologically active substances and biomaterials.

### Sequence-Listing Free Text

SEQ ID NO: 1 represents the base sequence of DNA encoding SurE (derived from wild-type Streptomyces albidoflavus NBRC 12854).

SEQ ID NO: 2 represents the amino acid sequence of SurE (derived from wild-type Streptomyces albidoflavus NBRC 12854).

SEQ ID NO: 3 represents the base sequence of a forward primer used for preparing a recombinant SurE.

SEQ ID NO: 4 represents the base sequence of a reverse primer used for preparing a recombinant SurE.

SEQ ID NO: 5 represents the amino acid sequence of recombinant SurE.

SEQ ID NO: 6 represents the base sequence of DNA encoding a TycC-TE (wild type Brevibacillus parabrevi ATCC 8185).

SEQ ID NO: 7 represents the amino acid sequence of TycC-TE (wild type Brevibacillus parabrevi ATCC 8185). SEQ ID NO: 8 represents the base sequence of the synthetic nucleic acid used for preparing a recombinant TycC-TE.

SEQ ID NO: 9 represents the amino acid sequence of a recombinant TycC-TE (having His tag at the N terminal and C terminal).

SEQ ID NO: 10 represents the base sequence of a forward primer used for preparing a recombinant SurE mutant (SurE (G235L)).

SEQ ID NO: 11 represents the base sequence of a reverse primer used for preparing SurE (G235L).

SEQ ID NO: 12 represents the amino acid sequence of SurE (G235L).

SEQ ID NO: 13 represents the base sequence of DNA encoding WolJ (wild type, Streptomyces sp. MST-110588).

SEQ ID NO: 14 represents the amino acid sequence of WolJ (wild type, Streptomyces sp. MST-110588).

SEQ ID NO: 15 represents the base sequence of a synthetic nucleic acid used for preparing recombinant WolJ.

SEQ ID NO: 16 represents the amino acid sequence of a recombinant WolJ (having a His tag at the N terminal).

The invention claimed is:

1. A method for producing a cyclic peptide, comprising using a penicillin-binding protein-type thioesterase (PBP-type TE) or a tyrocidine synthase TycC thioesterase domain (TycC-TE) as a catalyst, wherein a diol as a leaving group is attached to a carboxyl group of a C-terminal residue of a substrate.

2. The method according to claim 1, wherein the leaving group is ethylene glycol (EG) or an analogue thereof.

3. The method according to claim 2, wherein the leaving group is EG.

4. The method according to claim 1, wherein the PBP-type TE is used as the catalyst.

5. The method according to claim 4, wherein the PBP-type TE is an enzyme having an amino acid sequence represented by SEQ ID NO: 2 or a mutant enzyme thereof, and wherein the mutant enzyme has any one of the following amino acid sequences:
(a) an amino acid sequence having an identity of 38% or more to the amino acid sequence represented by SEQ ID NO: 2;
(b) an amino acid sequence having a substitution, deletion, insertion or addition of one, several or several tens of amino acids in the amino acid sequence represented by SEQ ID NO: 2; or
(c) an amino acid sequence encoded by a base sequence that hybridizes with a base sequence complementary to a base sequence represented by SEQ ID NO: 1 under a stringent condition,
and has a peptide cyclization activity equivalent to or higher than that of the enzyme having the amino acid sequence represented by SEQ ID NO: 2.

6. The method according to claim 4, wherein the PBP-type TE is an enzyme having an amino acid sequence represented by SEQ ID NO: 14 or a mutant enzyme thereof, and wherein the mutant enzyme has any one of the following amino acid sequences:
(a) an amino acid sequence having an identity of 35% or more to the amino acid sequence represented by SEQ ID NO: 14,
(b) an amino acid sequence having a substitution; deletion, insertion or addition of one, several or several tens of amino acids in the amino acid sequence represented by SEQ ID NO: 14; or
(c) an amino acid sequence encoded by a base sequence that hybridizes with a base sequence complementary to a base sequence represented by SEQ ID NO: 13 under a stringent condition,
and has a peptide cyclization activity equivalent to or higher than that of the enzyme having the amino acid sequence represented by SEQ ID NO: 14.

7. The method according to claim 1, wherein the TycC-TE is used as the catalyst.

8. The method according to claim 1, wherein the TycC-TE is an enzyme having an amino acid sequence represented by SEQ ID NO: 7 or a mutant enzyme thereof, and wherein the mutant enzyme has any one of the following amino acid sequences:
(a) an amino acid sequence having an identity of 35% or more to the amino acid sequence represented by SEQ ID NO: 7;
(b) an amino acid sequence having a substitution, deletion, insertion or addition of one, several or several tens of amino acids in the amino acid sequence represented by SEQ ID NO: 7; or
(c) an amino acid sequence encoded by a base sequence that hybridizes with a base sequence complementary to a base sequence represented by SEQ ID NO: 6 under a stringent condition,
and has a peptide cyclization activity equivalent to or higher than that of the enzyme having the amino acid sequence represented by SEQ ID NO: 7.

9. The method according to claim 1, wherein the substrate is obtained by a synthesis method comprising the following steps:
(i) elongating a peptide from a diol carried on a solid phase; and
(ii) cleaving the peptide to which the diol is bound from the solid phase.

10. The method according to claim 8, wherein the diol is EG or an analogue thereof.

11. The method according to claim 9, wherein the diol is EG.

12. A kit for producing a cyclic peptide, comprising a PBP-type TE or a TycC-TE.

13. The kit according to claim 11, further comprising a substrate having a diol as a leaving group attached to a carboxyl group of a C-terminal residue thereof, or a means for producing the substrate.


SEQUENCE LISTING (16 sequences)

SEQ ID NO: 1 - DNA, 1353 bp, Streptomyces albidoflavus
  source 1..1353: mol_type = genomic DNA
gtgggtgccgagggggcggaacgggacgcggtcggcgcgctcttcgaggagctggtgcgcgagcaccgggtcaccggggcgcagctgtccgtctaccgcgacggcgccctcagcgagtacgccaccggcctcgcctcggtccgcaccggtgaaccggtcaccccccggacgggtttccccttcggctcggtgaccaagttcctcaccgccgagctggtcatgcagttcgtctgcgacggcgacctggacctcgacgatcccctcgccgggctcctccccgacctggggcgcgccgccggcccgcccctcggcaccgccaccgtccgccagctcctcagccacaccgccggggtggtggacagcatcgagtacgacgagatgcgcggtccctcctaccggcggttcgccgcggcgtgcgcccggcagcccgcgctcttcccgccgggcctcgccttctcctactccaacaccgggtactgcctgctgggcgcggtgatcgaggcggcgtcggggatggactggtggacggcgatggacagttgcctgctgcgtccgctcggcatcgagccggccttcctgcacgacccgcgccccggccagggcggcgccgcccggccggtggccgagggccacgcgctgcgcgccggcggcgagcgggccgagcacgtcgaccacatggcctcgctctcgctggccgccgccggcgggctggtcggcagcgccaccgacctggtcaccgcggcccgcccgcacctggccgaccggaagaccttcgcccagcacgacctgctccccgaggacgccgtcctcgccatgcgcacctgcgtgccggacgccgagccgttcggcctggccgacggctgggggctgggcctgatgcgccacggcaccggcgacggcgcctggtacggccacgacggcgcggtgggcggcgcctcctgcaacctccgtatccacccggaccgttcgctggcgctggcgctgaccgccaactccaccgccgggccgaagctgtgggaggcgctggtcgcgcggctgccggaggccgggctcgacgtgggccactacgcgctgcccgtccccgactccgcgccgctggccccggacgccggccacctcggcacctacgccaacggcgacctggagctgatggtgacccacgacgccgccggcgacctcttcctgacccgcgagagctactccgactaccgcctctccctgcacgaggacgacctcttcgtggcccggagcggcgagcccggcgcgctcccgatcaccggccgcttcgtccgggagcacccggccgggccggtcgccctgctccagtacggcggccgggccatgcaccggctc

SEQ ID NO: 2 - protein, 451 aa, Streptomyces albidoflavus
VGAEGAERDAVGALFEELVREHRVTGAQLSVYRDGALSEYATGLASVRTGEPVTPRTGFPFGSVTKFLTAELVMQFVCDGDLDLDDPLAGLLPDLGRAAGPPLGTATVRQLLSHTAGVVDSIEYDEMRGPSYRRFAAACARQPALFPPGLAFSYSNTGYCLLGAVIEAASGMDWWTAMDSCLLRPLGIEPAFLHDPRPGQGGAARPVAEGHALRAGGERAEHVDHMASLSLAAAGGLVGSATDLVTAARPHLADRKTFAQHDLLPEDAVLAMRTCVPDAEPFGLADGWGLGLMRHGTGDGAWYGHDGAVGGASCNLRIHPDRSLALALTANSTAGPKLWEALVARLPEAGLDVGHYALPVPDSAPLAPDAGHLGTYANGDLELMVTHDAAGDLFLTRESYSDYRLSLHEDDLFVARSGEPGALPITGRFVREHPAGPVALLQYGGRAMHRL

SEQ ID NO: 3 - DNA, 30 bp, synthetic construct
  misc_feature 1..30: note = A nucleotide sequence of a forward primer (with EcoRI recognition site) for amplifying a DNA fragment encoding enzyme SurE.
  source 1..30: mol_type = other DNA
ccggaattccatatgggtgccgagggggcg

SEQ ID NO: 4 - DNA, 27 bp, synthetic construct
  misc_feature 1..27: note = A nucleotide sequence of a reverse primer (with HindIII recognition site) for amplifying a DNA fragment encoding enzyme SurE.
  source 1..27: mol_type = other DNA
cccaagctttcagagccggtgcatggc

SEQ ID NO: 5 - protein, 471 aa, synthetic construct
  REGION 1..471: note = An amino acid sequence of a recombinant enzyme SurE (with a histidine tag) obtained in the working example.
MGSSHHHHHHSSGLVPRGSHMGAEGAERDAVGALFEELVREHRVTGAQLSVYRDGALSEYATGLASVRTGEPVTPRTGFPFGSVTKFLTAELVMQFVCDGDLDLDDPLAGLLPDLGRAAGPPLGTATVRQLLSHTAGVVDSIEYDEMRGPSYRRFAAACARQPALFPPGLAFSYSNTGYCLLGAVIEAASGMDWWTAMDSCLLRPLGIEPAFLHDPRPGQGGAARPVAEGHALRAGGERAEHVDHMASLSLAAAGGLVGSATDLVTAARPHLADRKTFAQHDLLPEDAVLAMRTCVPDAEPFGLADGWGLGLMRHGTGDGAWYGHDGAVGGASCNLRIHPDRSLALALTANSTAGPKLWEALVARLPEAGLDVGHYALPVPDSAPLAPDAGHLGTYANGDLELMVTHDAAGDLFLTRESYSDYRLSLHEDDLFVARSGEPGALPITGRFVREHPAGPVALLQYGGRAMHRL

SEQ ID NO: 6 - DNA, 762 bp, Brevibacillus parabrevis
  source 1..762: mol_type = genomic DNA
acgcataagcgctttgagagcagatacggcacagccattttgttaaatcaggagacggcgcgaaacgtattttgcttcacgccgatcggcgcacaaagcgtgtactaccagaagcttgcggcggaaattcaaggcgtctctttgtacagctttgatttcatccaggatgacaaccggatggagcagtatatcgcggcgatcaccgcaattgatccaagcggtccgtacacgctcatgggctactcctcgggaggcaatctggcttttgaagtggcgaaagaactggaggagcggggctatggcgtcaccgacatcatcttgttcgactcgtactggaaagacaaggcgattgagcggactgtcgcggaaacagaaaacgacattgcccagctattcgccgagattggcgaaaacaccgagatgttcaacatgacgcaagaagacttccagctgtacgccgccaatgagtttgtcaagcaaagcttcgttcgcaaaacggtcagctatgtgatgttccataacaatctggtcaataccggaatgaccactgccgcgatccacctcatccaatccgagctggaagcagacgaggaagctccggtggcagccaagtggaacgaatcagcctgggcaaacgcaacgcaacgactgctgacgtacagcgggcacggaatccactcgcgcatgctggcgggcgattacgcgtcgcaaaatgcttcgattttgcaaaacatcctgcaagaactgttcatcctgaaataa

SEQ ID NO: 7 - protein, 253 aa, Brevibacillus parabrevis
THKRFESRYGTAILLNQETARNVFCFTPIGAQSVYYQKLAAEIQGVSLYSFDFIQDDNRMEQYIAAITAIDPSGPYTLMGYSSGGNLAFEVAKELEERGYGVTDIILFDSYWKDKAIERTVAETENDIAQLFAEIGENTEMFNMTQEDFQLYAANEFVKQSFVRKTVSYVMFHNNLVNTGMTTAAIHLIQSELEADEEAPVAAKWNESAWANATQRLLTYSGHGIHSRMLAGDYASQNASILQNILQELFILK

SEQ ID NO: 8 - DNA, 810 bp, synthetic construct
  misc_feature 1..810: note = A nucleotide sequence of a synthesized nucleic acid used for preparation of a recombinant TycC-TE
  source 1..810: mol_type = other DNA
catatgggtggcagtcacaaacgttttgaatctcgctatggcacggcgattctgcttaatcaggagacggctcgcaacgtcttctgttttaccccaatcggagcacagtcagtgtattatcagaaattagccgcagaaatccagggagttagcttatattcgttcgacttcatccaagacgataatcgcatggagcaatacattgcggctatcacggccattgatccctcaggtccatataccttaatgggctattctagcggtggcaacttggcgttcgaagtagcgaaagagctggaggagcgtgggtatggcgtgactgacatcattctgtttgattcgtactggaaggataaagccattgaacgcactgtcgcagagacggagaacgatattgcccagttatttgcggaaatcggcgaaaacactgaaatgttcaatatgacccaggaagactttcaattgtacgctgctaacgaatttgttaaacagtcatttgttcgtaagacagtctcttacgtgatgtttcataataatctcgttaacaccggtatgactactgcagccatccatctgattcaatccgagcttgaagctgacgaagaagccccggttgccgccaaatggaacgagtccgcgtgggccaacgccacacaacgtctgctgacctatagcggccatgggattcactcccgcatgctggcgggtgattacgcgtcgcagaacgcgagtatcctgcagaacattctgcaggaactgtttatcctgaaaggttcgcgcagccatcatcatcatcaccactaaaagctt

SEQ ID NO: 9 - protein, 286 aa, synthetic construct
  REGION 1..286: note = An amino acid sequence of the recombinant TycC-TE
MGSSHHHHHHSSGLVPRGSHMGGSHKRFESRYGTAILLNQETARNVFCFTPIGAQSVYYQKLAAEIQGVSLYSFDFIQDDNRMEQYIAAITAIDPSGPYTLMGYSSGGNLAFEVAKELEERGYGVTDIILFDSYWKDKAIERTVAETENDIAQLFAEIGENTEMFNMTQEDFQLYAANEFVKQSFVRKTVSYVMFHNNLVNTGMTTAAIHLIQSELEADEEAPVAAKWNESAWANATQRLLTYSGHGIHSRMLAGDYASQNASILQNILQELFILKGSRSHHHHHH

SEQ ID NO: 10 - DNA, 32 bp, synthetic construct
  source 1..32: mol_type = other DNA
  misc_feature 1..32: note = A nucleotide sequence of a forward primer for amplifying a DNA fragment encodingSurE(G235L).
tcgctggccgccgccctggggctggtcggcag

SEQ ID NO: 11 - DNA, 32 bp, synthetic construct
  source 1..32: mol_type = other DNA
  misc_feature 1..32: note = A nucleotide sequence of a reverse primer for amplifying a DNA fragment encodingSurE(G235L).
ctgccgaccagccccagggcggcggccagcga

SEQ ID NO: 12 - protein, 471 aa, synthetic construct
  source 1..471: note = An amino acid sequence of SurE(G235L).
MGSSHHHHHHSSGLVPRGSHMGAEGAERDAVGALFEELVREHRVTGAQLSVYRDGALSEYATGLASVRTGEPVTPRTGFPFGSVTKFLTAELVMQFVCDGDLDLDDPLAGLLPDLGRAAGPPLGTATVRQLLSHTAGVVDSIEYDEMRGPSYRRFAAACARQPALFPPGLAFSYSNTGYCLLGAVIEAASGMDWWTAMDSCLLRPLGIEPAFLHDPRPGQGGAARPVAEGHALRAGGERAEHVDHMASLSLAAALGLVGSATDLVTAARPHLADRKTFAQHDLLPEDAVLAMRTCVPDAEPFGLADGWGLGLMRHGTGDGAWYGHDGAVGGASCNLRIHPDRSLALALTANSTAGPKLWEALVARLPEAGLDVGHYALPVPDSAPLAPDAGHLGTYANGDLELMVTHDAAGDLFLTRESYSDYRLSLHEDDLFVARSGEPGALPITGRFVREHPAGPVALLQYGGRAMHRL

SEQ ID NO: 13 - DNA, 1380 bp, Streptomyces sp.
  source 1..1380: mol_type = genomic DNA; note = A nucleotide sequence of a DNA encoding a wild-type WolJ.
atgtctgcggaagcgaagggtcgcggtaccaccatcagcctgcccctcgaccgcctcgccaaagagcactccgtcgtcggcggccaactggccgtccaccaccaaggaaccactcacacctgggccttcggcgaggaggagcacgacagcggccggccggtcagcgtcggcaccgccttcgccctcggctcgaccaccaaagcactgaccgcgacctccgtcctccagctcgtctccgacggcgacctggacctggacagccccgtgaagacctggatcgaggagcgcggcgcggcggcggaccaccccgccctgaacgccacgctgcgccaactcctcagccacaccgccgggttgccctccgaccatttcgacgaggaagcgccctcgctgcgccgctggctggccgggttcctcacctccggtgacgagcgcgcccgcgcgtcctggcccacgcccggctccttctcgtactccaacatcggctacgccgtcgccggccgcgccgtggaggccgccaccggactgccctggtggcaggcggtccgcgactacctgctgacgccgctgggcaccggcatcggcctgctgcccggcgccccccaggacggccggccgctgaccgcggcccccgggcacaccgcccacaccacccccgacggccggacggtggtgcacgtcgtccagagcaccaccgacgcgggctcggcgccctccggcggcctcgcgggcagcgccgccgacctcgtacgtttcgcccggctgcacctggaggagcccggcgacctggaccgctccgccgtcgccgacccgtccgtattgcgcgagatggcccagcccgtcaccggcgccgaacccttcgggctggccgacggctggggcctgggcctgggccacttcggccccgccggccgccactggctgggccacgacggcaccctggacggcagcacctgtcacctgcgcatcgagccgcggcagggcaccgtcgtcgccctgaccaccaacacctcctcggggctggccctttgggactccgtcgtcgaggagctgcgcgagtgcggcctggacatcggcctgcacgcaccgcagccgccgctggccgcgcccgccgcgaccttcgcggagtgtacgggcacctaccgcaacggggacctggccgtgacggtcggcatcgaggaccagcacatcgtcctgggactgcccaacggccagcgggactccgtcgccccgcacaccggcctgttcttctccggccggcccggcaacgccagcctcttcctcggccggttcgtcaaggacgtggccaccggtgagatccgtgccctgcagtactccggacgcacgctgacacgcgaggcgctcacggccaggaactga

SEQ ID NO: 14 - protein, 459 aa, Streptomyces sp.
  source 1..459: note = An amino acid sequence of a wild-type WolJ.
MSAEAKGRGTTISLPLDRLAKEHSVVGGQLAVHHQGTTHTWAFGEEEHDSGRPVSVGTAFALGSTTKALTATSVLQLVSDGDLDLDSPVKTWIEERGAAADHPALNATLRQLLSHTAGLPSDHFDEEAPSLRRWLAGFLTSGDERARASWPTPGSFSYSNIGYAVAGRAVEAATGLPWWQAVRDYLLTPLGTGIGLLPGAPQDGRPLTAAPGHTAHTTPDGRTVVHVVQSTTDAGSAPSGGLAGSAADLVRFARLHLEEPGDLDRSAVADPSVLREMAQPVTGAEPFGLADGWGLGLGHFGPAGRHWLGHDGTLDGSTCHLRIEPRQGTVVALTTNTSSGLALWDSVVEELRECGLDIGLHAPQPPLAAPAATFAECTGTYRNGDLAVTVGIEDQHIVLGLPNGQRDSVAPHTGLFFSGRPGNASLFLGRFVKDVATGEIRALQYSGRTLTREALTARN

SEQ ID NO: 15 - DNA, 1413 bp, synthetic construct
  source 1..1413: mol_type = other DNA; note = A nucleotide sequence of a synthesized nucleic acid used for preparation of a recombinant WolJ.
atgaatcacaaagtgcatcatcatcatcatcatatgtctgccgaagctaagggtcgcggaacaactatttctttgcctctcgaccggttagccaaggagcactccgtcgtcggcgggcaacttgccgtgcaccatcaaggcacgacgcatacatgggccttcggggaagaggagcatgacagtggtcgtccagtgagtgtgggaacggcgttcgctttaggttctacgactaaggcactgaccgcaacatcggtgttgcaattggtttccgatggcgatttggacctggactcccctgtcaagacgtggatagaagaaagaggtgccgccgccgaccacccagcattaaacgcaacacttagacaacttttaagccacacagccgggctcccgtcagatcacttcgacgaagaggccccgagcctgcgccgctggctggcgggattcctgacaagtggcgacgagcgcgcacgtgcttcatggcctacacctggttcattttcgtactccaatattgggtacgccgttgccggtcgtgctgttgaagcggcaactggcttaccgtggtggcaggccgtgcgcgattacctcttgaccccgctggggacggggatcggtcttctccctggtgctccacaggacggccgtccattaacagcggctcctggccacacagcgcatacaacccctgacggtcgcactgtggtacacgtggtacaatctacaacagacgctggctcggccccgtcgggtggattggccgggtccgctgcagaccttgtgcgtttcgcgcgacttcacctggaagaacctggagatttagatcgttcagcggttgccgatcctagtgtgcttcgcgagatggcccaacctgtgaccggtgcagagccgttcggtcttgccgacggctggggtttaggcttaggccactttggaccggctgggcgtcattggctggggcatgacggcactcttgatggcagtacttgtcatttgcgaattgagccgcgtcaaggcacagtcgtggccttaacaacaaacacgagtagcggactggcactgtgggacagcgttgtagaggagttgcgtgagtgtggcctggacattggactccacgcaccgcaaccgcctctggcagctcctgcagcaacatttgccgagtgcaccggtacctaccggaatggtgatcttgcggtcactgtaggcatcgaagaccaacacattgtgcttggattgccgaacggccagcgcgacagtgttgctccgcacacgggtttgttcttctcaggccgtccgggaaacgcatctttattcttgggccgcttcgtcaaggacgtggcgacaggggagatccgtgctctccaatactccggccgcaccttgacccgcgaggccctgactgcgcgcaactaa

SEQ ID NO: 16 - protein, 470 aa, synthetic construct
  source 1..470: note = An amino acid sequence of a recombinant WolJ.
MNHKVHHHHHHMSAEAKGRGTTISLPLDRLAKEHSVVGGQLAVHHQGTTHTWAFGEEEHDSGRPVSVGTAFALGSTTKALTATSVLQLVSDGDLDLDSPVKTWIEERGAAADHPALNATLRQLLSHTAGLPSDHFDEEAPSLRRWLAGFLTSGDERARASWPTPGSFSYSNIGYAVAGRAVEAATGLPWWQAVRDYLLTPLGTGIGLLPGAPQDGRPLTAAPGHTAHTTPDGRTVVHVVQSTTDAGSAPSGGLAGSAADLVRFARLHLEEPGDLDRSAVADPSVLREMAQPVTGAEPFGLADGWGLGLGHFGPAGRHWLGHDGTLDGSTCHLRIEPRQGTVVALTTNTSSGLALWDSVVEELRECGLDIGLHAPQPPLAAPAATFAECTGTYRNGDLAVTVGIEDQHIVLGLPNGQRDSVAPHTGLFFSGRPGNASLFLGRFVKDVATGEIRALQYSGRTLTREALTARN